(19) United States
(12) Patent Application Publication
(10) Pub. No.: US 2007/0286881 A1
(43) Pub. Date: Dec. 13, 2007

(54) Method, composition and kit for treating degenerated disc disease and discogenic pain

(76) Inventor: Brian Burkinshsw, Pflugerville, TX (US)

Correspondence Address: ANDREWS KURTH LLP;Intellectual Property Department, Suite 1100, 1350 I Street, NW, Washington, DC, 20005, US

(21) Appl. No.: 11/802,642

(22) Filed: May 24, 2007

Related U.S. Application Data
(63) Continuation-in-part of application No. 11/181,677, filed on Jul. 14, 2005.

(51) Int. Cl. A61K 47/42; A61K 31/5415; A61K 9/08; A61K 38/48

(52) U.S. Cl. 424/422; 424/94.64; 514/226.2

(57) ABSTRACT

A method, and a corresponding kit and a pharmaceutical composition, is effective for treating discogenic pain caused by a degenerated disc. The method includes injecting a neurotropic agent and polymeric carrier, such as a fibrin sealant, into the degenerated disc. The fibrin sealant injected into the disc includes fibrinogen and an activating compound. The neurotropic agent may be injected before, simultaneously with, or after the injection of the fibrin sealant.

### RELATED APPLICATION

This application is a continuation-in-part of application Ser. No. 11/181,677, filed Jul. 14, 2005, and entitled Enhanced Biological Autologous Tissue Adhesive Composition and Methods of Preparation and Use, the disclosure of which is hereby incorporated by reference in its entirety.

### BACKGROUND

Degenerative disc disease is one of today's most common and costly medical conditions. Marked by the gradual erosion of cartilage and disc degeneration between the vertebrae, this destructive spinal disease routinely provokes discogenic pain, especially in the lower back. Discogenic pain affects about 80 percent of the population some time during their lives and, in the United States, is the leading cause of disability in people under age 45 (see, e.g., Andersson G B, Acta Orthop Scand Suppl, 1999, 69:28-31).

The pathogenesis of degenerative disc disease is poorly understood. The factors that account for the vulnerability of the disc to degeneration and the limited capacity of the disc for repair remain largely unknown. However, recent studies suggest that the in-growth of vascularized granulation tissue along torn fissures in the disc may be the source of discogenic pain (see, e.g., Peng et al. Spine, 2006, 31:560-566).

Traditional treatments for degenerative disc disease and pain include local injection of anti-inflammatory medications, such as steroids, or the use of non-steroid anti-inflammatory drugs (NSAIDs), physical therapy, behavior modifications, intradiscal electrothermal therapy (IDET) and surgical interventions. Each of these treatments has disadvantages. For example, physicians use steroids to suppress inflammation and the resulting chemical and physical pain associated with such inflammation. However, epidural steroid injection (ESI) currently is considered to be a treatment only for radicular pain, and not discogenic pain. Furthermore, steroid use also comes with side effects, not the least of which is steroid's effect in the body's immune system and the suppression of the body's ability to fight off infection.

### DESCRIPTION OF THE DRAWINGS

The detailed description will refer to the following drawings in which:

FIG. 1 is a cross-sectional view of a vertebral body at the disk space exhibiting annular fissures that may be treated according to the herein disclosed embodiments;

FIG. 2 illustrates an embodiment of a delivery device for injecting fluids into a spinal disc to treat degenerative disc disease and discogenic pain; and

FIG. 3 illustrates a kit used for treating degenerative disc disease and discogenic pain.

### DETAILED DESCRIPTION

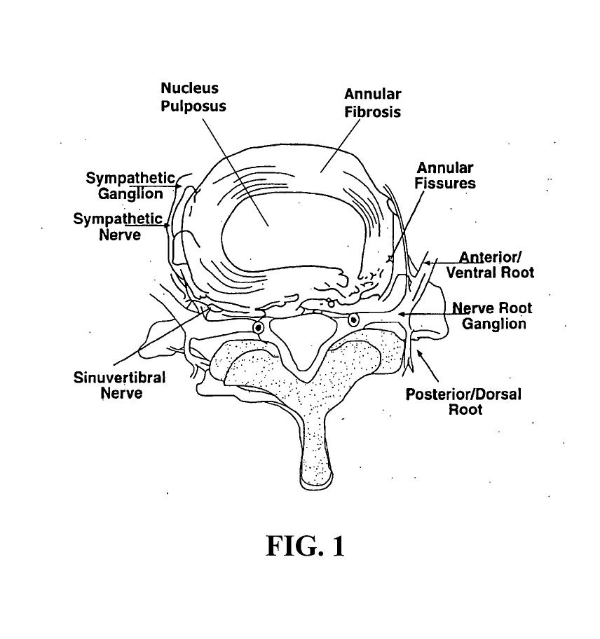

Recent clinical studies suggest that innervation of a degenerated disc is one possible source of discogenic pain. Innervation occurs following an annular injury, tear or significant disruption in the annulus fibrosus of the disc (see FIG. 1). When this disc injury occurs, the normal healing process attempts to lay down new tissues to seal the wound. However, instead of the annular wound healing from outside to inside and stopping at the inner border of the annulus fibrosus, a tendency exists for granulation tissues to continue to form along the fissures, past the inner border of the annulus fibrosus, and into the center of the nucleus pulposus. These granulation tissues bring immuno-reactive nerve fibers and microscopic blood vessels into the nucleus pulposus. Since the post-pubescent nucleus pulposus is essentially an avascular environment, with a slightly acidic pH, it does not provide a normal or supportive environment for normal nerve in-growth. The result is what may be termed “rogue” nerves, which are the likely source of discogenic pain.

Previous attempts to treat discogenic pain include the direct injection of a neurotropic agent into the disc in an effort to deaden or destroy the rouge nerves that have formed in the disc nucleus. However effective such disc injections may be in reducing discogenic pain, they do not treat the underlying defect (i.e., the fissures) that allowed the in-growth of the rouge nerves. Furthermore, with the continued existence of fissures, fluids, such as the neurotopic agents, can leak out of the intradiscal space. Such leakage of the neurotropic agent can reduce its effectiveness in treating discogenic pain, and also runs the risk of destroying nerve endings outside the affected disc volume.

To address these and other shortcomings of current discogenic pain treatment methods, novel resorbable, natural biologic matrix, disc augmentational repair methods, and corresponding pharmaceutical compositions and kits are disclosed. One embodiment of the aforementioned methods, compositions and kits involves administering a neurotropic agent and a polymeric carrier. The polymeric carrier may be, for example, a fibrin sealant.

Neurotropic agents, as disclosed herein, may destroy the rogue nerve endings that have formed in the disc nucleus or inner ⅔ of the annulus, allowing the disc to heal in a more normal scarring fashion. In the enclosed, anaerobic environment of the disc, a relatively small dose of a well-understood neurotropic, if it could be made to persist for an extended period before dispersing, would prevent premature regeneration of new nerve tissues into the disc before healing or scarring can take place in and around the fissures of the annulus. By preventing in-growth of rogue nerves and the resulting inflammation they generate, the pain and inflammatory mediators should abate and a more natural healing process should occur. In one embodiment, the herein disclosed fibrin sealant creates a matrix that facilitates localized delivery of the neurotropic agent by restricting the administration of the neurotropic agent at the treatment site and by modulating the release of the neurotropic agent over time.

The matrix also seals the fissures of the annulus and prevents the disc from leaking material from the nucleus into the area outside the disc. Furthermore, sealing halts the leakage of harmful chemicals from the disc environment and prevents the initiation of immune responses towards the damaged disc. Moreover, the injection of the neurotropic agent and the fibrin sealant, at least temporarily, may provide a bulking effect, which in turn may increase the spacing between lamina, which then relieves pressure on the nerve roots passing through the intervertebral foramen and hence may reduce radicular pain.

The neurotropic agent can be any agent that is capable of blocking nerve conduction, inhibiting nerve growth, causing apoptosis of neuronal cells, or devitalizing a nerve fiber. Examples of neurotropic agents include, but are not limited to, methylene blue, phenol, phenyl combined with glycerine, ethyl alcohol, hypertonic saline, ammonium salt solutions, chlorocresol, botox, batroxobin, various viper snake venoms, and Aloe Vera extracts. A single neurotropic agent, or a mixture of two or more neurotropic agents, may be used.

The fibrin sealant is formed from fibrinogen and an activating agent that converts fibrinogen to fibrin. Fibrinogen can be autologous (i.e., from the patient to be treated), heterologous (i.e., from other human, pooled human supply, or non-human source such as bovine and fish), or recombinant. Fibrinogen can be fresh or frozen. Fibrinogen is commercially available in freeze-dried form. Freeze-dried fibrinogen is commonly reconstituted in a solution containing aprotinin (a polyvalent protease inhibitor that prevents premature degradation of the formed fibrin). In one embodiment, the reconstitution solution contains aprotinin at a concentration of 3000 KIU/ml.

The activating agent can be any agent that causes fibrinogen to form fibrin. Examples of the activating agent include, but are not limited to, thrombin and enzymes derived from arachnid venom or snake venom, such as batroxobin. Thrombin is an enzyme that converts fibrinogen to fibrin. Thrombin can be autologous, heterologous, or recombinant. Thrombin can be fresh or frozen. Thrombin is commercially available in freeze-dried form. Freeze-dried thrombin can be reconstituted in water or water containing calcium ions. In one embodiment, the reconstitution solution contains calcium chloride in the range of about 1 to 100 mmol/ml.

Fibrin sealants act as barriers or “fillers” as well as tissue adhesives, and thus the pore size of the fibrin sealant macrostructure scaffold may actually inhibit the formation of granulation tissues, depending on the concentrations of fibrinogen, thrombin and fibronectin present during the clotting process. Assuming there are sufficient quantities of fibrinogen and thrombin present to produce a fibrin matrix smaller than 150 microns, new cell formation of granular (scar) tissue would not be able to penetrate the matrix until the clot degraded.

In addition to acting as a neurotropic agent, certain agents may also cause the formation of fibrin when mixed with the fibrinogen. For example, and as noted above, one technique to produce fibrin is by means of a thrombin-like enzyme, which includes thrombin. A thrombin-like enzyme is any enzyme that can catalyze the formation of fibrin from fibrinogen. A common source of activating agent (the thrombin-like enzyme) is a snake venom. Other sources of agents that serve the dual purpose of activating agent and neurotropic agent include various venomous marine life, such as jellyfish, sea snakes, cone shells, and sea urchins. Preferably, the thrombin-like enzyme is purified from the venom (e.g., from snake venom). Depending on the choice of activating agent, such thrombin-like enzyme can release fibrinopeptide A—which forms fibrin I—fibrinopeptide B—which forms des BB fibrin—or both fibrinopeptide A and B—which forms fibrin II. Activating agents that release fibrinopeptide A and B may do so at different rates. Thus, the resultant composition could be, for example, a mixture of fibrin II and fibrin I or a mixture of fibrin II and des BB fibrin.

Table I is a nonlimiting list of the sources of the snake venoms that can be used with the herein disclosed methods, compositions, and kits, the name of the thrombin-like enzyme, and which fibrinopeptide(s) is released by treatment with the enzyme.

TABLE 1

| | | Fibrinopeptide |
|---|---|---|
| Source | Name | Released |
| Agkistrodon acutus | Acutin | A |
| A. contortrix contortrix | Venzyme | B, (A)* |
| A. halys pallas | | B, (A)* |
| A. (Calloselasma) | Ancrod, Arvin | A |
| rhodostoma | | |
| Bothrops asper | Asperase | A |
| B. atrox, B. moojeni, | Batroxobin | A |
| B. maranhao | | |
| B. insularis | Reptilase | A, B |
| B. jararaca | Botropase/bothrombin | A |
| Lachesis muta muta | Defibrase | A, B |
| Crotalus adamanteus | Crotalase | A |
| C. durissus terrificus | | A |
| Trimeresurus flavoviridis | Flavoxobin/habutobin | A |
| T. gramineus | Grambin | A |
| Bitis gabonica | Gabonase | A, B |

*( ) means low activity.

For a review of thrombin-like enzymes from snake venoms, see H. Pirkle and K. Stocker, Thrombosis and Haemostasis, 65(4):444-450 (1991).

The preferred thrombin-like enzymes are Batroxobin, especially from B. moojeni, B. maranhao and B. atrox; and Ancrod, especially from A. rhodostoma.

In one embodiment of the herein disclosed methods, an affected disc is injected with fibrin sealant intradiscally to seal the fissure(s), followed by an intradiscal injection of the neurotropic agent to destroy or deaden rogue nerves in the disc. In another embodiment, the disc is injected intradiscally first with the neurotropic agent to destroy or deaden the rogue nerves, followed by an intradiscal injection of fibrin sealant to subsequently seal the fissure(s) and prevent any new in-growth of nerves. In yet another embodiment, the neurotropic agent is infused, mixed or combined with the fibrin sealant, and injected simultaneously into the affected disc. Once injected into the affected disc, the fibrin sealant acts as a carrier for the neurotropic agent, and the neurotropic agent slowly leaches out of the fibrin sealant matrix to deaden or destroy nerves within the now-sealed disc space.

In yet another embodiment, the neurotropic agent is a snake venom having thrombin-like enzyme activities, such as batroxobin or Asperase. The neurotropic agent is injected into the disc, followed by an intradiscal injection of fibrinogen, preferably in the presence of Ca++ ions. The neurotropic agent acts to destroy or deaden rogue nerves in the disc. Following the injection of the fibrinogen, the neurotropic agent assumes a secondary, or dual, role of activating fibrinogen to form fibrin. The now-sealed disc then is free of active rogue nerves and would has the benefit of the fibrin healing matrix.

In the herein disclosed methods, fibrin formation begins immediately on contact of the fibrinogen and the activating agent, such as occurs in a Y-connector of a dual syringe injection device. One such dual syringe injection device is described in U.S. Patent Application Ser. No. 60/854,413, which is hereby incorporated by reference in its entirety.

The term “injecting” fibrin sealant as used herein thus encompasses any injection of components that form fibrin in the disc, including circumstances where a portion of the components react to form fibrin due to mixing prior to contact with or actual introduction into the disc. The herein disclosed methods include the sequential injection of the components of the fibrin sealant into the disc, such as by injecting the activating agent followed by the fibrinogen, or by injecting the fibrinogen followed by the activating agent. Likewise, the fibrinogen and the activating agent each can be intermittently injected into the disc. The neurotropic agent may be pre-mixed with either the activating agent or the fibrinogen, and injected as described above. Alternatively, the neurotropic agent may be injected separately before or after the injection of the fibrin sealant.

Fibrin sealants mimic the final stage of the natural clotting mechanism. Typically, such sealants entail the mixing of a fibrinogen component with an activating enzyme such as thrombin. To increase biocompatibility of the sealant with host tissue, various components may be supplied endogenously from host body fluids. Combining the reconstituted components produces a viscous solution that quickly sets into an elastic coagulum. A method of preparing a conventional fibrin sealant is described by J. Rousou, et al. (J. Rousou, et al. Journal of Thoracic and Cardiovascular Surgery, 1989, 97:194-203). Cryoprecipitate derived from source plasma is washed, dissolved in a buffer solution, filtered and freeze-dried. The freeze-dried fibrinogen is reconstituted in solution containing a fibrinolysis inhibitor. The solution is stirred and heated to a temperature of about 37° C. Each solution (the thrombin and fibrinogen solutions) is drawn up in a syringe and mounted on a Y-connector to which a needle is attached for delivery of the combined solution (see, e.g. the Duploject™ device, from ImmunoAG, Vienna, Austria). Thus, mixing of the components only occurs during the delivery process, which facilitates clot formation only at the desired site of application. The components should be injected sufficiently quickly to avoid the passage becoming blocked due to fibrin formation in the needle and/or Y-connector.

In one embodiment, a dual-syringe injector is used and the mixing of the fibrin sealant components at least partially occurs in the Y-connector and in the needle mounted on a Y-connector, with the balance of the clotting occurring in the disc. This method of preparation facilitates the formation of a fibrin clot at the desired site in the disc during delivery, or immediately thereafter.

In another embodiment, the apparatus for delivering fibrin sealant includes a delivery device and a pressure monitor. The pressure monitor couples to the delivery device through a line connected to a transducer operably attached to a reservoir such as, for example, being operably attached to one of the syringes. Alternatively, the transducer can be located within the connector, or anywhere else where the transducer can be introduced within the device such that pressure of fluid within the device can be measured. The pressure monitor can be mechanical, but is typically an electronic monitor with a digital readout such as through a liquid crystal display (LCD) built into the housing.

In one embodiment, the delivery device includes at least two reservoirs for fluids such as a multi-barrel syringe, a pressure monitor, an introducer needle, a fluid delivery tube adapted to receive fluid from a first barrel of the multi-barrel syringe and adapted to extend into the introducer needle, and a connector coupled to a second barrel of the multi-barrel syringe, wherein the connector is coupled to the introducer needle and adapted to receive the fluid delivery tube so that the fluid delivery tube extends into the introducer needle.

In other embodiments, the fluid delivery tube can be a needle or a catheter. In one embodiment, the fluid delivery tube attaches directly to a syringe, such as by way of a luer fitting. Alternatively, the fluid delivery tube may be integral with the connector. For example, the connector can be made by forming the connector around a portion of the needle in an injection molding process or other process.

Pressure monitors are available commercially. For example, pressure monitors are currently available from Merit Medical Systems, Inc. (Utah) sold as a Meritrans™ transducer. Other representative pressure monitors are disclosed in, for example, U.S. patent application number 2005/0004518, incorporated herein by reference. In such a device, a pressure transducer is integrally mounted in the plunger of a syringe under the plunger tip such that the force applied by the plunger to the fluid in the syringe is transmitted to the transducer and the resulting electronic signal is converted to a display value, aiding the physician in diagnosing diseased disks in the back. The transducer of the pressure monitor can be positioned in the barrel of a syringe or, alternatively, in the connector.

The apparatus for delivering fibrin sealant also includes fluid reservoirs (such as a multi-barrel syringe), a connector, a fluid delivery tube, and an introducer needle. The syringe, connector, and needle can be coupled using standard luer fittings. The fluid reservoirs can include handles and plungers. Alternatively, the fluid reservoirs can be configured such that the reservoirs are flexible and can be squeezed or rolled to force fluids out. The introducer needle can, for example, couple to the connector by a luer fitting at an end connector opposite to the end connected to the syringe. The fluid from the barrel is driven through a fluid delivery tube that has been pushed through a plug attached to or integral with the connector, with the fluid delivery tube being of sufficient length to be threaded into the introducer needle. In one embodiment, the fluid delivery tube couples to a first barrel of a multi-barrel syringe and the fluid delivery tube extends into the connector through a plug coupled to the connector. In one embodiment, the fluid delivery tube directly couples to the first barrel of the syringe, and the fluid delivery tube is affixed to the connector so that the fluid delivery tube cannot move within the introducer needle. Fluid from the barrel is pushed through a conduit within the connector and flows into the introducer needle. Thus, the connector is adapted for conveying fluid from the fluid delivery tube into the introducer needle. The connector can include a passage for fluid from the second barrel to the introducer needle, with the passage being of a diameter such that the fluid from the second syringe barrel is of a volume approximately equal to the volume of fluid delivered through the fluid delivery tube. In one embodiment, the fluid delivery tube is of a length such that it does not protrude out the end of the introducer needle. The fluids from barrel mix near the distal tip of the introducer needle. The pressure monitor is attached to a transducer such that the transducer of the pressure monitor is within the barrel to measure internal pressure within the barrel. The pressure measured within the barrel will be the same or nearly the same pressure as that at the distal tip of the introducer needle during a procedure. Thus, the pressure monitor allows the pressure within the disc to be monitored. In one embodiment, the multi-barrel syringe has two barrels. Each barrel can be configured to couple to the connector or fluid delivery tube by a luer fitting. A delivery device of this invention may be equipped with a trip switch if a given pressure is reached, which reduces the chance of an over-pressurized disc.

In another configuration of the delivery device, the fluid delivery tube is integral with the connector so that the fluid delivery tube does not need to be inserted through a plug. The fluid delivery tube can be bonded to the connector or can be otherwise coupled to the connector so that fluid from the barrel flows into the fluid delivery tube. A first fluid, such a fibrinogen, is injected through either the fluid delivery tube or through the conduit, with the activating compound being injected through the opposite passage from that used by the fibrinogen. Thus the tow fluids flow through the device in coaxially and do not tough or mix until the given fluid exits the fluid delivery tube.

A wide variety of designs can be used for the fluid delivery device. For example, the device can include a delivery gun equipped with a ratcheting lever to make injection easier. Such a delivery gun could also be automated so that physical pressure is not needed by the physician in order for injection to proceed. In use, the gun could be loaded with the multiple barrels that contain the fibrinogen and activating compound liquids. Compression of the lever would force plungers to push the fluids from out of the barrels and into the connector, fluid delivery tube, and/or introducer needle. Alternatively, the gun could use a screw-type action to move the plungers. Either embodiment gives the physician a mechanical advantage when injecting the components.

In another embodiment, freeze-dried fibrinogen is reconstituted to a concentration of about 75-115 mg/ml, and freeze-dried thrombin is reconstituted separately to a final concentration of about 400-600 IU/ml. Freeze-dried fibrinogen and freeze-dried thrombin are available in kit form from such manufacturers as Baxter under names such as TISSEEL™. These two fibrin sealant components can be prepared in about 2 ml samples each to yield approximately 4 ml of total sealant (reconstituted fibrinogen plus reconstituted thrombin). In another embodiment, at least one of the reconstituted fibrinogen and thrombin is reconstituted using a solution containing at least one additive. In yet another embodiment, at least one of the reconstituted fibrinogen and thrombin is reconstituted using a solution containing at least one neurotropic agent. A preservative-free reconstituting solution may be used, but is not required.

The neurotropic agent and fibrin sealant disclosed herein may be injected into the disc, at the zygapophysical joint (also called Z-joint or facet joint), the costovertebral joints (articulation of the rib with the vertebral body), or the sacroiliac joint. For the treatment of a degenerated disc, the neurotropic agent and fibrin sealant are injected into the nucleus pulposus of the affected disc, shown in FIG. 1, to fill any fissures or voids of the annulus fibrosus, seal the bone end plates to the disc, increase pressure of the disc, at least temporarily acting as a bulking agent and hence increase the height of the disc space. In general, the neurotropic agent and fibrin sealant are injected at a location near the defect in the annulus fibrosus so that the neurotropic agent and fibrin sealant will flow into the fissures in the annulus fibrosus. Since the intended purpose of neurotropic agent preferentially is to disrupt or destroy the rogue nerve endings that form in the disc nucleus following an annular injury, tear or significant disruption that occurs in the annulus fibrosus of the disc, great care should be taken not to expose normal nerves to the neurotropic agent.

The point, or points, of injection (e.g., at the tip of a spinal needle) can be within the annulus fibrosus or in the nucleus pulposus. If the injection occurs in the nucleus pulposus, the injected components may form a patch at the interface between the nucleus pulposus and the annulus fibrosus, or, more commonly, the components flow into the defect(s) (e.g., fissures) of the annulus fibrosus and potentially overflowing into the interdiscal space. Over-pressurizing the disc when injecting the components into the disc should be avoided.

The neurotropic agent and/or fibrin sealant may be administered with an anesthetic, such as a local anesthetic. Representative examples of such local anesthetics include but are not limited to lidocaine HCL (often sold in concentrations of 1.5 percent or 4 percent), SARAPIN anesthetic (a sterile aqueous solution of soluble salts and bases from Sarraceniaceae (Pitcher Plant), and bupivacaine HCL (also known as marcaine, which is often sold in concentrations of 0.5 percent and 0.75 percent). The chemical name for lidocaine is alpha-diethylaminoaceto-2,6-xylidide, and the IUPAC name is 2-(diethylamino)-N-(2,6-dimethylphenyl)acetamide. The chemical name for bupivicaine is 1-butyl-N-(2,6-dimethylphenyl)-2-piperidinecarboxamide, sometimes referred to as 1-butyl-2′,6′-pipecoloxylidide monohydrochloride, having registry number 14252-80-3. Alternatively, procaine (2-diethylaminoethyl 4-aminobenzoate hydrochloride) or other local anesthetic can be employed. Among the local anesthetics, bupivacaine is preferred. Combinations of anesthetics also can be used. The anesthetic can be injected with the neurotropic agent or the fibrin sealant, if the neurotropic agent and the fibrin sealant are injected sequentially. The anesthetic can be injected with the neurotropic agent and the fibrin sealant, if the neurotropic agent and the fibrin sealant are injected simultaneously. Alternatively, the anesthetic can be injected separately, either before or after the neurotropic agent and/or fibrin sealant have been injected. In an embodiment, the anesthetic is injected prior to, or simultaneously with, the injection of the neurotropic agent and/or the fibrin sealant.

In one embodiment, a solution containing a local anesthetic is used to reconstitute the fibrinogen, the activating agent, or the neurotropic agent. In another embodiment, one of the fibrinogen, the activating agent, or the neurotropic agent is reconstituted without an anesthetic, and the anesthetic is then added to the reconstituted fibrinogen, the activating agent, or the neurotropic agent.

In general, the amount of anesthetic used should be chosen so as to be effective in alleviating the pain of injection when the sealant is injected or otherwise introduced into the disc. In one embodiment, a solution containing about 0.1 to about 10 percent by weight of anesthetic is used. The injected volume of the anesthetic solution can vary widely, such as from about 0.1 ml to about 5 ml, depending on the mode of injection.

The neurotropic agent and/or fibrin sealant may be administered with one or more additives. As used herein, the term additives includes antibiotics; antiproliferative, cytotoxic, and antitumor drugs including chemotherapeutic drugs; analgesic; antiangiogen; antibody; antivirals; cytokines; colony stimulating factors; proteins; chemoattractants; chelating agent such as EDTA; histamine; antihistamine; erythropoietin; antifungals; antiparasitic agents; non-corticosteroid anti-inflammatory agents; anticoagulants; anesthetics including local anesthetics such as lidocaine and bupivicaine; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans such as aggrecan (chondrotin sulfate and deratin sulfate), versican, decorin, and biglycan; antiangiogenins; antigens; DBM; hyaluronic acid and salts and derivatives thereof; polysaccharides; cellulose compounds such as methyl cellulose, carboxymethyl cellulose, and hydroxy-propylmethyl cellulose and derivatives thereof; antibodies; gene therapy reagents; genetically altered cells, stem cells including mesenchymal stem cells with transforming growth factor, and/or other cells; cell growth factors to promote rehabilitation of damaged tissue and/or growth of new, healthy tissue such as BMP7 and BMP2; type I and II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage including autologous cartilage; oxygen-containing components; enzymes such as, for example, peroxidase, which mediate the release of oxygen from such components; melatonin; vitamins; and nutrients such as, for example, glucose or other sugars. In one embodiment, the additive is a growth factor that promotes rehabilitation of the damaged tissues.

Any of the aforementioned additives may be added to the neurotropic agent or fibrin sealant separately or in combination. For example, one or more of these additives can be injected with the fibrin sealant. Alternatively, one or more of these additives can be injected with the neurotropic agent, if the neurotropic agent is injected separately, either before or after the fibrin sealant has been injected. Combinations of these additives can be employed and different additives can be used in the solutions that are used to reconstitute the fibrinogen, the activating agent, or the neurotropic agent. In one embodiment, a solution containing a local anesthetic is used to reconstitute the fibrinogen, a solution containing type II collagen is used to reconstitute the activating agent, and a solution containing glucosamine sulfate is used to reconstitute the neurotropic agent. Likewise, one or more of these additives can be injected with the fibrin sealant or the neurotropic agent. Alternatively, one or more of these additives can be injected separately, either before or after the fibrin sealant and/or the neurotropic agent have been injected.

For solutions containing an incompletely water-soluble additive(s), an anti-caking agent such as polysorbate may be added to facilitate suspension of this component.

The neurotropic agent and the fibrin sealant, or compositions thereof, will generally be used in an amount effective to achieve the intended result, i.e., ameliorating discogenic pain and other symptoms of a degenerative disc disease. The compound(s) may be administered therapeutically to achieve a therapeutic benefit. As used herein, a therapeutic benefit means the eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of neurotropic agent and the fibrin sealant to a patient suffering from a degenerative disc disease provides a therapeutic benefit not only when the underlying degenerative disc disease is eradicated or ameliorated, but also when the patient reports a decrease in the severity or duration of the symptoms associated with the degenerative disc disease. A therapeutic benefit also includes halting or slowing the progression of the disease, regardless of whether improvement is realized.

The amount of the agents administered will depend upon a variety of factors, including, for example, the particular indication being treated, the mode of administration, whether the desired benefit is prophylactic or therapeutic, the severity of the indication being treated and the age and weight of the patient, and the bioavailability of the particular agent. Determination of an effective dosage is well within the capabilities of those skilled in the art.

Effective dosages may be estimated initially from in vitro assays and in vivo animal models. For example, an initial dosage of neurotropic agent for use in animals may be formulated to achieve a local concentration that would effectively inhibit neuronal cell growth based on in vitro data. Calculating dosages to achieve such concentrations taking into account the bioavailability of the particular compound is well within the capabilities of skilled artisans. Guidance for calculating such doses is provided in Fingl & Woodbury, “General Principles,” In: Goodman and Gilman's The Pharmaceutical Basis of Therapeutics, Chapter 1, pp. 1-46, Pagamonon Press, and the references cited therein, all of which are hereby incorporated by reference.

Suitable animal models of degenerative disc diseases and discogenic pain include rat and rabbit models described in, for example, Norcross et al., An in vivo model of degenerative disc disease, J. Orthopaedic Research, 2003, 21:183-188; and Larson et al., Biologic Modification of Animal Models of Intervertebral Disc Degeneration, The Journal of Bone and Joint Surgery (American), 2006, 88:83-87. Ordinarily, skilled artisans can routinely adapt such information to determine dosages suitable for human administration.

The data obtained from cell culture assays and animal studies can be used in formulating a range of dosages for use in humans. The dosage of such compounds may lie within a range of concentrations that exhibit an ED₅₀ with little or no toxicity. The dosage may vary within this range depending upon the dosage form employed and the route of administration utilized. For any neurotropic agent used according to the disclosed method, a therapeutically effective dose can be estimated initially from cell culture assays. A dose may be formulated in animal models to achieve a dose range that exhibits an IC₅₀ (i.e., the concentration of the test neurotropic agent which achieves a half-maximal inhibition of neuronal cells) as determined by cell culture assays. The effects of any particular dosage can be monitored by suitable assays. In humans, the effect of the neurotropic agent and/or fibrin sealant on pain and function can be measured by the visual analog scale (VAS) or Morris Roland Disability Index.

Dosage amounts of the neurotropic agent will typically be in the range of from about 0.01 mg to about 1.5 mg per injection of 1 percent methylene blue. In one embodiment, the neurotropic agent is methylene blue prepared as a 0.1-5.0 percent (w/v) solution in water or other suitable solvent and is injected in a dose range of 0.2-50 mg per injection.

The fibrinogen is typically used in a concentration range of 25 to 150 mg/ml. The amount of activating agent such as thrombin can be varied to reduce or lengthen the time to complete fibrin formation. The fibrinogen is typically in the range 50 to 150 mg/ml and the thrombin in the range 4 IU/ml to 600 IU/ml. In general, the higher level of thrombin per unit amount of fibrinogen, the faster fibrin formation occurs. If slower fibrin formation is desired, less thrombin is used per unit fibrinogen. The fibrin formation time (i.e., the polymerization time of the fibrinogen) may be important for controlling the time at which the clot forms so as to ensure the fibrin sealant sets up at the proper site and time in the body rather than setting-up prematurely. Likewise, varying the fibrinogen concentration may change the density of the combined components, which may be important for controlling flow through a long conduit such as a catheter into the body. The use of calcium ions (such as from calcium chloride) in one or both of the component solutions will affect the strength of the fibrin so formed, with increasing amounts of calcium ions increasing the strength of the fibrin clot.

Because of the restricted space within a disc, the total volume of the injection is limited. Typically, 0.2 to 1 ml of neurotropic agent and 1 to 4 ml of fibrin are used for sequential intradiscal injections, and a total volume of 1 to 5 ml is used for simultaneous intradiscal injections (neurotropic agent and fibrin sealant).

The dosage, injection volume, and injection interval may be adjusted individually to provide local concentrations of the agents that are sufficient to maintain a therapeutic benefit. For example, the neurotropic agent and fibrin sealant may be administered simultaneously in a single injection, or by sequential injections. The neurotropic agent may be injected minutes, hours, or days before or after the injection of the fibrin sealant. The injection may be repeated periodically. Skilled artisans will be able to optimize effective local dosages and the injection regimen without undue experimentation.

Preferably, the neurotropic agent and fibrin sealant will provide a therapeutic benefit without causing substantial toxicity. Toxicity of the neurotropic agent and/or fibrin sealant may be determined using standard pharmaceutical procedures. The dose ratio between toxic and therapeutic effect is the therapeutic index. Agents that exhibit high therapeutic indices are preferred.

A contrast agent may be used in conjunction with the injection of the neurotropic agent and/or fibrin sealant to ensure the correct placement at the site and avoidance of blood vessels. The contrast agent may be injected prior to injection of the neurotropic agent and/or fibrin sealant. Alternatively, the contrast agent may be included in the fibrinogen component or the activating agent component, or the neurotropic agent that is injected into the disc. Contrast agents and their use are well known to those skilled in the art.

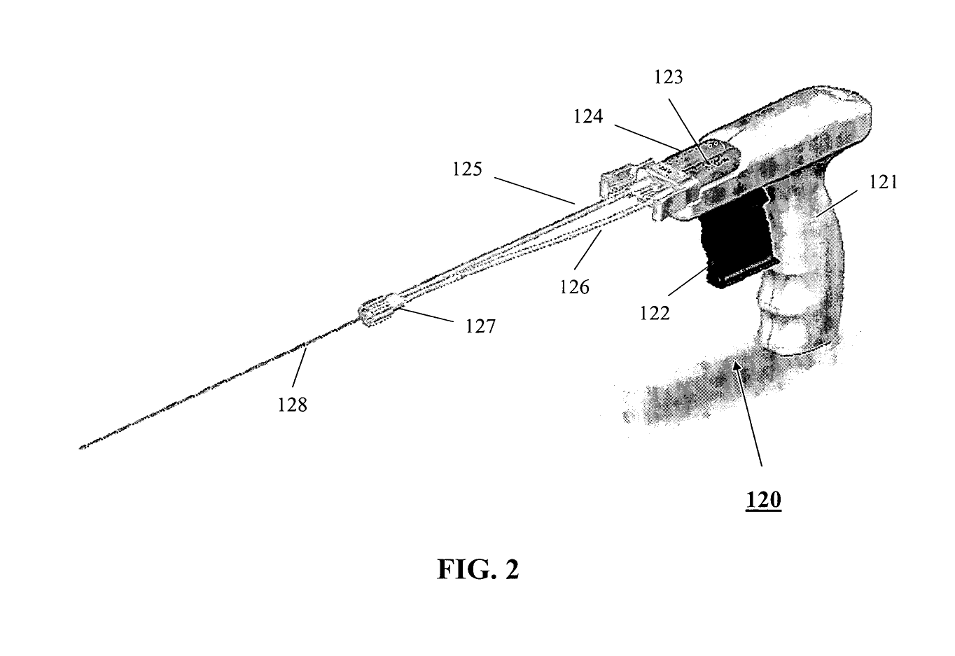

The neurotropic agent and fibrin sealant may be injected into the disc using a delivery device such as that shown in FIG. 2. Delivery device 120 includes main housing 121 into which are inserted fibrinogen capsule 123 and thrombin capsule 124. Trigger 122, in conjunction with a pressure monitor (not shown), controls injection of the fluids. Attached to the capsules 123, 124 is an inner needle assembly including delivery tubes 125 and 126. Connector 127 serves to connect the delivery tubes 125, 126 to a coaxial intradiscal needle 128.

In addition to the disclosed methods, also disclosed is a pharmaceutical composition for treating degenerative disc diseases. The pharmaceutical composition includes a neurotropic agent, a fibrinogen, an activating agent, a pharmaceutically acceptable carrier, and optionally one or more additives.

As used herein a “pharmaceutically acceptable carrier” is intended to include any and all solvents, solubilizers, stabilizers, bases, buffering agents, controlled release vehicles, diluents, emulsifying agents, dispersion media, antibacterial or antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary agents can also be incorporated into the compositions.

The pharmaceutical composition is formulated to be compatible with its intended route of administration. Examples of routes of administration include injection into the intradiscal space, the zygapophysical joint, the costovertebral joints, and the sacroiliac joint. Solutions or suspensions used for the injection can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Sterile injectable solutions can be prepared by incorporating the neurotropic agent and/or fibrin sealant components in the required amount in an appropriate solvent with one or a combination of ingredients enumerated above, as required, followed by filtered sterilization. Generally, dispersions are prepared by incorporating the active compound into a sterile vehicle that contains a basic dispersion medium and the other required ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Because fibrin formation begins immediately on contact of fibrinogen and the activating agent, the fibrinogen and the activating agent of the pharmaceutical composition are kept in different containers and are mixed during injection using, for example, the delivery device 120 of FIG. 2.

In another embodiment, the fibrinogen and the activating agent are kept in separate containers. The fibrinogen and the activating agent may be mixed during injection using specially designed dual-syringe injection device, with a coaxial needle or multi-lumen needle.

In one embodiment, the neurotropic agent is methylene blue and the activating agent is thrombin and Ca++ ions.

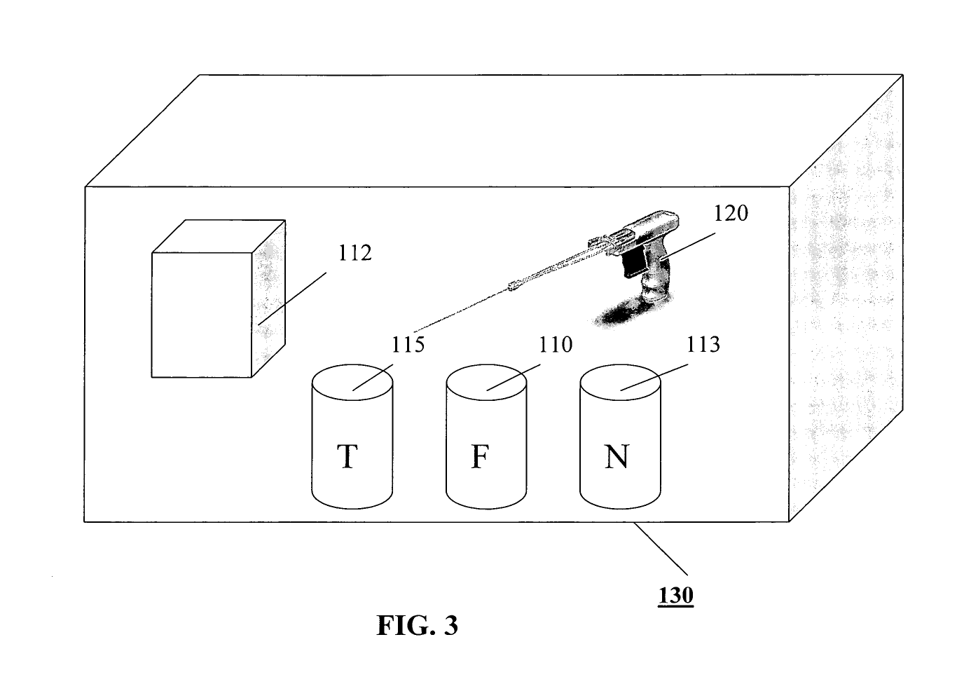

In addition to the above-described methods and pharmaceutical compositions, also disclosed is a kit for treating degenerative disc disease and discogenic pain with neurotropic agents and fibrin sealants. FIG. 3 shows a representative kit. The kit 100 includes fibrinogen 110, an activating compound 115, and a fibrin sealant delivery device 120 (shown assembled for clarity—the delivery device 120 is packaged as individual components in the kit 100) for injecting fibrin sealant into a human disc, wherein the apparatus is equipped with a pressure monitor. The kit 100 may be stored and shipped in a suitable container 130. The kit 100 may include additional items, such as but not limited to, a neurotropic agent 113, one or more additives, a source of calcium ions, a device 112 for reconstituting freeze-dried fibrinogen, additional fluid delivery tubes, and additional intradiscal needles.

In one embodiment, the kit comprises a neurotropic agent, a fibrinogen, an activating agent, and at least one reconstituting solution. The fibrinogen and the activating agent are kept in separate containers. The neurotropic agent may be kept in another separate container.

In another embodiment, the kit further comprises an anesthetic. The anesthetic may be a local anesthetic such as lidocaine, sarapin or bupivicaine.

In another embodiment, the kit further comprises one or more additives.

In another embodiment, the kit further comprises a spinal needle or a polymeric catheter or both.

In another embodiment, the kit further comprises a dual syringe injector.

In yet another embodiment, the kit further comprises an instruction.

Use of the improved fibrin sealant composition may be better understood by reference to the following example, which is representative and should not be construed to limit the scope of the claims hereof. Unless otherwise indicated, the procedures are conducted in the absence of a heating step of the nucleus fibrosus and annulus fibrosus and in the absence of a partial or total discectomy.

### EXAMPLE

### Intradiscal Injection of Methylene Blue and Fibrin Sealant

Injection of the fibrin sealant and neurotropic agent (e.g., methylene blue) involves several steps, which are outlined below. The example presented is based on use of the delivery device 120 shown in FIG. 2.

Pre-Medication

As a first step, intravenous antibiotics are administered 15 to 60 minutes prior to commencing the procedure as prophylaxis against discitis. Patients with a known allergy to contrast medium should be pre-treated with H1 and H2 blockers and corticosteroids prior to the procedure in accordance with International Spine Intervention Society (ISIS) recommendations. Sedative agents may be administered but the patient should remain awake during the procedure and capable of responding to pain from pressurization of the disc.

Preparation

The injection procedure should be performed in a suite suitable for aseptic procedures and equipped with fluoroscopy (C-arm or two-plane image intensifier) and an x-ray compatible table to allow visualization of needle placement.

Local anesthetic for infiltration of skin and deep tissue and nonionic contrast medium with 10 mg per cc of antibiotic should be available for this procedure.

Preparation of the Fibrin Sealant and Methylene Blue Mixture

Preparation of the fibrin sealant and methylene blue mixture requires approximately 25 minutes. In an embodiment, freeze-dried fibrinogen and thrombin are reconstituted in a fibrinolysis inhibitor solution and a calcium chloride solution, respectively. Methylene blue may be freeze-dried and added by controlled mass measurements and distributed within either the fibrinogen or the thrombin, or it may be added in liquid form after reconstitution of the freeze-dried fibrinogen and thrombin. The dissolution within normal carrier liquids results in reconstituted fibrinogen and thrombin solutions (one of which also contains methylene blue). The solutions are then combined upon delivery with the delivery device 120 to form the fibrin sealant within the treated disc.

Preparation of the Delivery Device

Maintaining a sterile environment, the delivery device 120 is assembled with the thrombin and fibrinogen capsules inserted into the body of the device.

Patient Positioning and Skin Preparation

The patient should lie on a radiography table in either a prone or oblique position depending on the physician's preference. The skin of the lumbar and upper gluteal region should be prepared as for an aseptic procedure using non-iodine containing preparations.

Target Identification

Disc visualization and annulus fibrosus puncture should be conducted according the procedures used for provocation discography. The targeted disc should be approached from the side opposite of the patient's predominant pain. If the patient's pain is central or bilateral, the target disc can be approached from either side.

An anterior-posterior (AP) image of the lumbar spine is obtained such that the x-ray beam is parallel to the inferior vertebral endplate of the targeted disc. The beam should then be angled until the lateral aspect of the superior articular process of the target segment lies opposite the axial midline of the target disc. The path of the intradiscal needle should be parallel to the x-ray beam, within the transverse mid-plane of the disc, and just lateral to the lateral margin of the superior articular process.

Placement of the Intradiscal Needle

The intradiscal needle is specifically designed to facilitate annular puncture and intradiscal access for delivery of the fibrin sealant and the neurotropic agent. The intradiscal needle is manufactured with a slight bend in the distal end to enhance directional control of the needle as it is inserted through the back muscles and into the disc.

The intended path of the intradiscal needle is anesthetized from the subcutaneous tissue down to the superior articular process. The intradiscal needle initially may be inserted under fluoroscopic visualization down to the depth of the superior articular process. The intradiscal needle will be then slowly advanced through the intervertebral foramen while taking care not to impale the ventral ramus. If the patient complains of radicular pain or paraesthesia, advancement of the needle must be stopped immediately and the needle must be withdrawn approximately 1 cm. The path of the needle should be redirected and the needle slowly advanced toward the target disc. Contact with the annulus fibrosus will be noted as a firm resistance to continued insertion of the intradiscal needle. The needle will be then advanced through the annulus to the center of the disc. Placement of the needle is confirmed with both AP and lateral images. The needle tip should lie in the center of the disc in both views.

Once the needle position is confirmed, a small volume of nonionic contrast medium will be injected into the disc. A minimal volume of contrast will be injected to insure avascular flow of the contrast media. If vascular flow is seen, the intradiscal needle should be repositioned and the contrast injection repeated.

Loading the Delivery System

After correct placement of the intradiscal needle is confirmed, the reconstituted fibrinogen and thrombin solutions are transferred into the delivery device 120. Using aseptic techniques, the reconstituted fibrinogen is drawn into the fibrinogen syringe and thrombin into the thrombin syringe.

Attaching the Inner Needle Assembly and Intradiscal Needle

The inner needle assembly next is attached to the delivery device 120, and air is expelled from the device. The intradiscal needle is then attached.

Delivery of the Fibrin Sealant

Placement of the intradiscal needle tip in the center of the target disc is reconfirmed with AP and lateral images. The trigger is then depressed to begin application of fibrin sealant to the disc. Pressure should be monitored constantly when squeezing the trigger. To prevent over-pressurization of a (lumbar) disc, pressure should not exceed 100 psi (6.8 atm).

Each full compression of the trigger will deliver approximately 1 mL of the fibrin sealant/methylene blue mixture to the disc. When the trigger is released, it automatically resets to the fully uncompressed position. Once all of the fibrin sealant/methylene blue mixture has been delivered, the trigger will stop advancing.

Periodic images of the disc should be taken during application of the fibrin sealant/methylene blue mixture to insure that the intradiscal needle has not moved from the center of the disc.

Application of the fibrin sealant/methylene blue mixture to the disc should continue until one of the three following events occurs.
1. The total available volume of the fibrin sealant/methylene blue mixture is delivered to the disc.
2. Continued application of the fibrin sealant/methylene blue mixture (to a lumbar disc) would require pressures above 100 psi (6.8 atm).
3. The patient cannot tolerate continuation of the procedure.

After the application of the fibrin sealant/methylene blue mixture is stopped, the intradiscal needle is carefully removed from the patient. Patient observation and vital signs monitoring will be performed for about 20-30 minutes following the procedure.

The herein described methods, compositions, and kits may be used to address various conditions through use of the neurotropic agent and fibrin sealant. For example, the methods, compositions and kits may be used to treat diseases, and resulting pain, in thoracic and cervical disc areas.

The disclosure references particular means, materials and embodiments elaborating limited application of the claims. Although the claims make reference to particular means, materials and embodiments, it is to be understood that the claims not limited to these disclosed particulars, but extend instead to all equivalents.

What is claimed is:

1. A method of treating discogenic pain caused by a degenerated disc, comprising:
injecting an effective amount of a neurotropic agent into the degenerated disc; and
injecting an effective amount of a polymeric carrier into the degenerated disc.

2. The method of claim 1, wherein the polymeric carrier is fibrin sealant, and, wherein injecting an effective amount of the fibrin sealant comprises mixing fibrinogen and an activating agent, whereby the fibrin sealant is formed.

3. The method of claim 2, wherein the neurotropic agent is injected simultaneously with the fibrin sealant, and wherein the fibrinogen and the activating agent are mixed during injection.

4. The method of claim 2, wherein the neurotropic agent is injected simultaneously with the fibrin sealant, the method further comprising pre-mixing the neurotropic agent with one of the fibrinogen and the activating agent prior to the neurotropic agent injection.

5. The method of claim 4, wherein the activating agent is thrombin and the neurotropic agent is methylene blue.

6. The method of claim 2, wherein the neurotropic agent and the fibrin sealant are injected sequentially and wherein the neurotropic agent is injected first.

7. The method of claim 2, wherein the neurotropic agent destroys/disrupts rogue nerves and is the activating agent that activates the fibrinogen to form the fibrin sealant.

8. The method of claim 2, wherein the neurotropic agent and the fibrin sealant are injected sequentially and wherein the fibrin sealant is injected first.

9. The method of claim 2, further comprising injecting the disc with the neurotropic agent and the fibrin sealant at multiple sites.

10. The method of claim 2, wherein the activating agent is thrombin.

11. The method of claim 2, wherein the fibrinogen is autologous fibrinogen.

12. The method of claim 1, wherein the polymeric carrier is degradeable/biodegradeable.

13. The method of claim 1, wherein the polymeric carrier is non-degradeable/non-biodegradeable.

14. The method of claim 1, wherein the polymeric carrier, after injection, forms a matrix that provides for controlled release of the neurotropic agent.

15. The method of claim 1, wherein the neurotropic agent is selected from the group consisting of methylene blue, phenol, phenyl combined with glycerine, ethyl alcohol, hypertonic saline, ammonium salt solutions, chlorocresol, botox, batrxobin, various viper snake venoms, extracts or refinements thereof, Aloe Vera extracts, and mixtures thereof.

16. The method of claim 1, further comprising injecting an anesthetic with one of the neurotropic agent and the polymeric carrier.

17. The method of claim 16, wherein the anesthetic, polymeric carrier and the neurotropic agent are injected sequentially and wherein the anesthetic is injected first.

18. The method of claim 16, wherein the anesthetic is a local anesthetic.

19. The method of claim 18, wherein the local anesthetic is selected from the group consisting of lidocaine, saparin, and bupivacaine.

20. The method of claim 1, wherein the neurotropic agent or the fibrin sealant further comprises at least one additive.

21. The method of claim 20, wherein the additive is selected from the group consisting of antibiotics; antiproliferative, cytotoxic, and antitumor drugs including chemotherapeutic drugs; analgesic; antiangiogen; antibody; antivirals; cytokines; colony stimulating factors; proteins; chemoattractants; EDTA; histamine; antihistamine; erythropoietin; antifungals; antiparasitic agents; non-corticosteroid anti-inflammatory agents; anticoagulants; anesthetics; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans; antiangiogenins; antigens; DBM; hyaluronic acid and salts and derivatives thereof; polysaccharides; cellulose compounds and derivatives thereof; antibodies; gene therapy reagents; genetically altered cells, stem cells including mesenchymal stem cells with transforming growth factor, and/or other cells; cell growth factors; type II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage; oxygen-containing components; enzymes; melatonin; vitamins; and nutrients.

22. The method of claim 1, further comprising forming the fibrin sealant in the presence of aprotinin and calcium ions.

23. The method of claim 1, wherein the injection is performed using a dual syringe injector.

24. The method of claim 1, wherein the injection is performed using a coaxial needle.

25. The method of claim 1, wherein the injection is performed using a multi-lumen needle.

26. The method of claim 1, wherein the injection is performed using a multi-lumen catheter.

27. The method of claim 1, further comprising injecting the neurotropic agent and the fibrin sealant in volumes sufficient to restore normal hydrostatic pressure in the disc or normal disc height, or both.

28. The method of claim 1, wherein the injecting steps comprise:
inserting an introducer needle having a tip into an intradiscal space of the degenerated disc to a position adjacent to a defect;
inserting a second needle or a polymeric catheter through the introducer needle up to but not beyond the tip of the introducer needle; and
injecting the neurotropic agent and the fibrin sealant through the needle or catheter assembly.

29. The method of claim 1, wherein the degenerated disc is a lumbar disc.

30. The method of claim 1, wherein the degenerated disc is a cervical disc.

31. The method of claim 1, wherein the degenerated disc is a thoracic disc.

32. The method of claim 1, further comprising injecting a contrast agent into the degenerated disc before the injection of the neurotropic agent, the fibrin sealant, or a mixture of the neurotropic agent and the fibrin sealant.

33. The method of claim 1, wherein a contrast agent is injected into the degenerated disc with the neurotropic agent, the fibrin sealant, or a mixture of the neurotropic agent and the fibrin sealant.

34. A kit for treating discogenic pain caused by a degenerated disc, comprising:
a neurotropic agent;
a fibrinogen; and
an activating agent, wherein the fibrinogen and the activating agent are kept in different containers.

35. The kit of claim 34, further comprising an anesthetic.

36. The kit of claim 35, wherein the anesthetic is a local anesthetic selected from the group consisting of lidocaine, sarapin or bupivicaine.

37. The kit of claim 34, wherein at least one of the neurotropic agent, the fibrinogen, and the activating agent is in a freeze-dried form, a liquid form, a frozen form or combinations thereof.

38. The kit of claim 37, further comprising at least one reconstituting solution.

39. The kit of claim 38, wherein the activating agent is thrombin and wherein the at least one reconstitution solution comprises aprotinin, or calcium ions, or both.

40. The kit of claim 39, wherein the neurotropic agent comprises methylene blue.

41. The kit of claim 34, further comprising one or more additives selected from the group consisting of antibiotics; antiproliferative, cytotoxic, and antitumor drugs including chemotherapeutic drugs; analgesic; antiangiogen; antibody; antivirals; cytokines; colony stimulating factors; proteins; chemoattractants; EDTA; histamine; antihistamine; erythropoietin; antifungals; antiparasitic agents; non-corticosteroid anti-inflammatory agents; anticoagulants; anesthetics; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans; antiangiogenins; antigens; DBM; hyaluronic acid and salts and derivatives thereof; polysaccharides; cellulose compounds and derivatives thereof; antibodies; gene therapy reagents; genetically altered cells, stem cells including mesenchymal stem cells with transforming growth factor, and/or other cells; cell growth factors; type II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage; oxygen-containing components; enzymes; melatonin; vitamins; and nutrients.

42. The kit of claim 34, further comprising at least one of an introducer needle, a spinal needle, a polymeric catheter, a coaxial spinal needle, a multi-lumen spinal needle and a multi-lumen catheter.

43. The kit of claim 42, further comprising a dual syringe injector.

44. A pharmaceutical composition for treating degenerative disc diseases, comprising:
a neurotropic agent;
a fibrinogen;
an activating agent

45. The pharmaceutical composition of claim 44, further comprising an anesthetic.

46. The pharmaceutical composition of claim 44, further comprising one or more additives.

47. The pharmaceutical composition of claim 44, wherein the neurotropic agent is methylene blue, and the activating agent is thrombin.

48. The pharmaceutical composition of claim 47, further comprising aprotinin and calcium chloride.

49. A method of treating discogenic pain caused by a degenerated disc, comprising:
injecting an effective amount of a neurotropic agent into the degenerated disc; and
injecting an effective amount of a fibrin sealant into the degenerated disc, wherein injecting an effective amount of the fibrin sealant comprises mixing fibrinogen and an activating agent, whereby the fibrin sealant is formed, and wherein the neurotropic agent and the fibrin sealant are injected together in a single injection and the neurotropic agent also serves as the activating agent.

50. The method of claim 49, wherein the neurotropic agent is derived from snake venom.

51. The method of claim 50, wherein the neurotropic agent is batroxobin.

52. A method for treating degenerated disc disease and associated discogenic pain, comprising:
injecting an effective amount of a neurotropic agent into a degenerated disc;
injecting a resorbable, biologic carrier into the disc to form a matrix that seals fissures in the disc.
